(19) United States
(12) Patent Application Publication
(10) Pub. No.: US 2025/0072914 A1
(43) Pub. Date: Mar. 6, 2025

(54) SYSTEMS AND METHODS RELATING TO SURGICAL STAPLES AND SURGICAL STAPLE INSERTERS

(71) Applicant: GLOBUS MEDICAL, INC., AUDUBON, PA (US)

(72) Inventors: Chad Glerum, Pennsburg, PA (US); Jesse Rush, Wayne, PA (US); Richard Scheinfield, Philadelphia, PA (US); Garret Norton, Lima, OH (US); Justin Horst, Wayne, PA (US); Jessica Sandoe, Blue Bell, PA (US)

(21) Appl. No.: 18/754,475

(22) Filed: Jun. 26, 2024

Related U.S. Application Data
(63) Continuation-in-part of application No. 18/459,474, filed on Sep. 1, 2023.

(51) Int. Cl. A61B 17/17; A61B 17/064; A61B 17/56

(52) U.S. Cl. CPC A61B 17/17; A61B 17/0642; A61B 17/56; A61B 2017/0641; A61B 2017/564

(57) ABSTRACT

Devices, systems, and methods thereof for treating fractures of the foot using staples and a staple inserter. A drill guide for surgical implantation systems includes features using k-wires received through a drill guide head to assist with drilling of pilot holes. A staple inserter may have features which take advantage of k-wires used in connection with the drill guide. An associated tamp may be employed in conjunction with staple implantation procedures. Surgical staples may also have certain geometries to foster improved compression characteristics.

### CROSS REFERENCE TO RELATED APPLICATION(S)

This application is a continuation-in-part application of U.S. patent application Ser. No. 18/459,474, filed on Sep. 1, 2023, which is hereby incorporated by reference in its entirety for all purposes.

### FIELD OF THE INVENTION

The present disclosure relates to surgical implants for treating fractures of bones in the foot and more particularly, the present disclosure relates to surgical nitinol staples for implantation into the foot to treat bone fractures and inserters for implanting surgical nitinol staples into the foot.

### BACKGROUND OF THE INVENTION

During surgical procedures to treat a fracture on an area of the foot, surgeons may use screws or plates to secure the fracture. The use of screws and plates for fractures, arthrodesis or osteotomies can lead to loss of compression over time. Post-surgery, any micromovement of the bone fragments during patient mobility can result in movement and loosening of the implants. Malunion or nonunion can occur if too much compression is lost, and a revision surgery might be needed to correctly compress the bone fragments together. Therefore, there exists a need for alternative fixation systems and methods, such as staples and staple inserters, to overcome the above-noted deficiencies.

### SUMMARY OF THE INVENTION

To meet this and other needs, according to one embodiment, a hand-held drill guide may be used in a surgical staple procedure on a patient with a fracture. The drill guide includes a head with multiple bores extending through it. One set of bores is sized to slidably and rotatably receive a surgical drill bit therethrough. A second set of bores is spaced longitudinally and in the proximal direction from the first set of bores. This second set of bores is able to receive k-wires therethrough to assist in fixing or pinning the drill guide in a desired position, so that pilot holes may be drilled, for instance.

In other versions, a surgical implantation system may have a staple inserter with features adapted to take advantage of the aforementioned k-wires. For example, a pair of slots matching the spacing and offset of the k-wires relative to the drill guide holes may be used to receive k-wires which have been affixed to and extend from the implantation site.

In still other variations, certain staple configurations include two or more planar surfaces formed at their bridges to improve functionality, including increasing compression as compared to staple configurations with non-planar, constant arcuate bridges.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a surgical staple for implantation in bone according to an exemplary embodiment of the present disclosure.

FIG. 2 shows a surgical staple inserter according to an exemplary embodiment of the present disclosure.

FIG. 3 shows a surgical staple attached to an inserter for implantation in bone according to an exemplary embodiment of the present disclosure.

FIG. 4 shows a surgical staple attached to an inserter for implantation in bone according to an exemplary embodiment of the present disclosure.

FIG. 5 shows a surgical staple attached to an inserter for implantation in bone according to an exemplary embodiment of the present disclosure.

FIGS. 6A and 6B show a surgical staple for implantation in bone according to an exemplary embodiment of the present disclosure.

FIGS. 7A and 7B show a surgical staple for implantation in bone according to an exemplary embodiment of the present disclosure.

FIGS. 8A and 8B show a surgical staple for implantation in bone according to an exemplary embodiment of the present disclosure.

FIGS. 9A, 9B, and 9C show a surgical staple for implantation in bone according to an exemplary embodiment of the present disclosure.

FIGS. 10A, 10B, and 10C show a surgical staple for implantation in bone according to an exemplary embodiment of the present disclosure.

FIGS. 11A, 11B, and 11C show a surgical staple for implantation in bone according to an exemplary embodiment of the present disclosure.

FIGS. 12A, 12B, and 12C show a surgical staple for implantation in bone according to an exemplary embodiment of the present disclosure.

FIGS. 13A and 13B show a surgical staple inserter according to an exemplary embodiment of the present disclosure.

FIG. 14 shows an exploded view of a surgical staple inserter according to an exemplary embodiment of the present disclosure.

FIGS. 15A and 15B show a surgical staple inserter according to an exemplary embodiment of the present disclosure.

FIGS. 16A and 16B show a portion of a surgical staple inserter according to an exemplary embodiment of the present disclosure.

FIG. 17 shows a portion of a surgical staple inserter according to an exemplary embodiment of the present disclosure.

FIG. 18 shows a portion of a surgical staple inserter according to an exemplary embodiment of the present disclosure.

FIGS. 19A and 19B show a surgical staple inserter according to an exemplary embodiment of the present disclosure.

FIG. 20 shows an exploded view of a surgical staple inserter according to an exemplary embodiment of the present disclosure.

FIG. 21 shows a surgical staple inserter according to an exemplary embodiment of the present disclosure.

FIG. 22 shows a surgical staple inserter according to an exemplary embodiment of the present disclosure.

FIG. 23 shows a surgical staple inserter according to an exemplary embodiment of the present disclosure.

FIG. 24 is an isometric view of one possible implementation of a hand-held drill guide according to the present disclosure.

FIG. 25 is an exploded, isometric view of the hand-held drill guide of FIG. 24.

FIG. 26 is an isometric view of one possible implementation of a head portion of the hand-held drill guide of FIGS. 24 and 25.

FIG. 27 is an isometric view of another possible implementation of a head portion of the hand-held drill guide of FIGS. 24 and 25.

FIG. 28 is an isometric view of still another possible implementation of a head portion of the hand-held drill guide of FIGS. 24 and 25.

FIG. 29 is an isometric view of a proximal portion of another possible implementation of a hand-held drill guide according to the present disclosure.

FIG. 30 is an isometric view of one possible implementation of a staple inserter according to the present disclosure.

FIG. 31 is an isometric view of a possible implementation of a surgical staple according to the present disclosure.

FIG. 32 is an isometric view of another possible implementation of a surgical staple according to the present disclosure. FIG. 33 is a top plan view of the surgical staple of FIG. 32.

### DETAILED DESCRIPTION OF THE INVENTION

Embodiments of the disclosure are generally directed to devices, systems, and methods for fixation of fractures involving the foot. Nitinol staples are an alternative or complementary fixation to screw or plate fixation of a fracture, arthrodesis or osteotomy. The shape memory or superelastic properties of nitinol allow for the staples to provide continuous compression across the bone gap. The nitinol staples are designed with the legs bent inward in their free state. Prior to insertion during surgery, the staple legs are distracted outward using an inserter so that the legs are parallel or close to parallel. The staple is implanted and the inserter is removed, thereby allowing the legs to return to their free state, which will result in the continuous compression of the bone gap. Compression provided by staples is significantly less likely to loosen than compression provided by screws and/or plates due to the superelastic properties of nitinol. The following disclosure involves staple designs that include staples designed for forefoot, midfoot, hindfoot, and staples that provide greater rotational stability for applications that require more compression. The various sizes of each style will provide the surgeon with a full set that covers all applications for use in the foot.

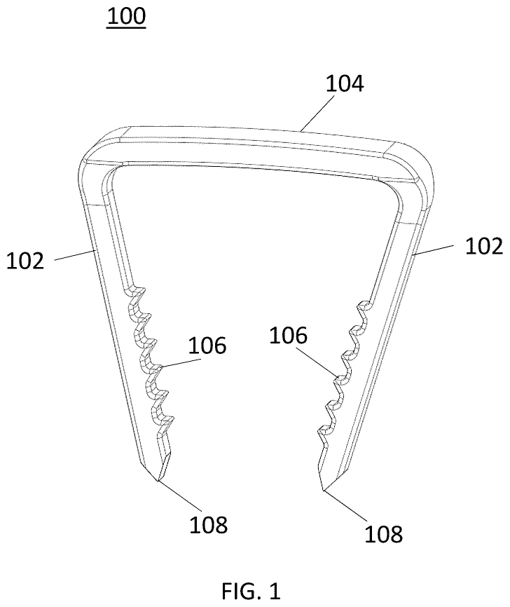

Turning to FIG. 1, an exemplary staple consistent with the principles of the present disclosure is illustrated. Staple 100 includes legs 102 and a bridge 104. Nitinol staples are designed with the legs bent inward in its free state. The traditional way of inserting these staples involves determining the bridge length and leg length of the staple, using a drill guide based on the size chosen and drilling parallel holes on either side of the bone gap that are normal to the surface of the bone. Then, the staple is loaded onto an inserter by distracting the legs of the staple outward so that the legs are parallel and implanting it into bone. The inserter is then removed from the staple, thereby allowing the legs to return to their free state, which will result in the continuous compression of the bone gap. These multiple instruments can either be reusable or single use but do add a little time to the procedure of inserting a nitinol staple.

A procedure involving the implantation of a nitinol staple can be burdensome due to the number of instruments used, and the challenges in maintaining visibility of the pre-drilled holes for the staple legs prior to insertion. Consistent with the principles of the present disclosure, a system and method of inserting a staple using ultrasonic technology is described in more detail below which allows a surgeon to insert a nitinol staple without the need for pre-drilling pilot holes for the staple legs.

The present disclosure allows different ways of inserting a nitinol staple using ultrasonic technology by allowing the staple to create its own path into bone without the need for a pre-drilled hole. By using ultrasonic technology, the sharp tips of the legs of the staple are able to cut through the bone while being inserted.

FIG. 1 illustrates an exemplary staple 100 having two legs 102. In between legs 102 is bridge 104. Bridge 104 and legs 102 could have different lengths depending on the size of staple chosen. Legs 102 may have teeth 106 which are used to prevent staple 100 from backing out of the bone once implanted. Legs 102 may have sharp tips 108 which are used to help facilitate insertion of the staple into bone.

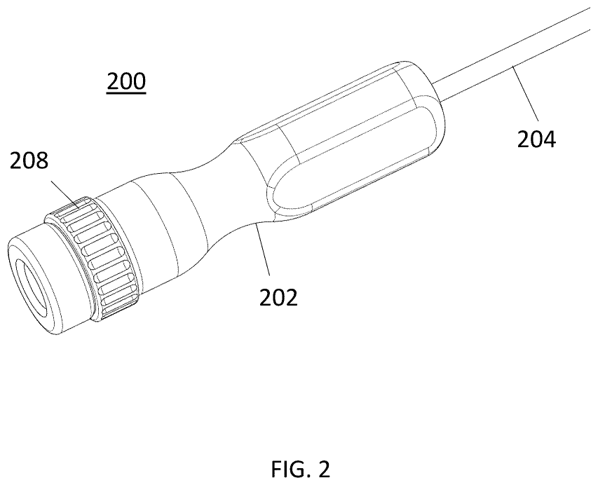

FIG. 2 illustrates an exemplary embodiment of an ultrasonic device 200 consistent with the principles of the present disclosure. Ultrasonic device 200 includes a handle 202 connected to a cord 204 which supplies electrical current from a power supply to ultrasonic device 200. The power supply provides motion to components inside handle 202.

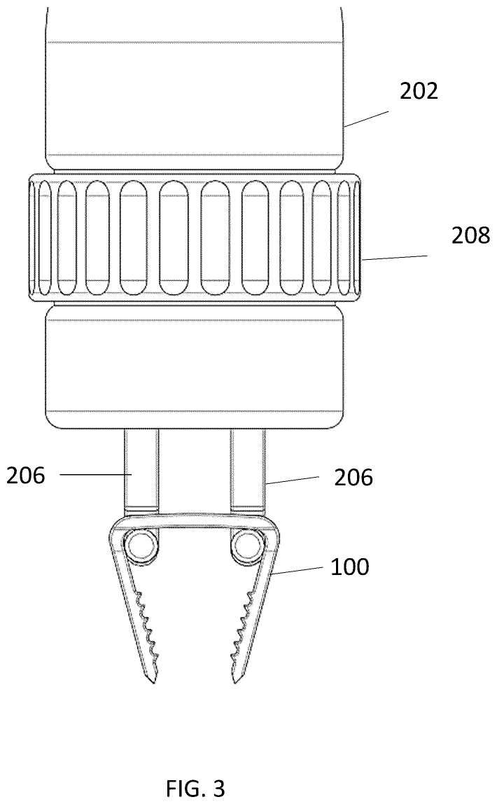

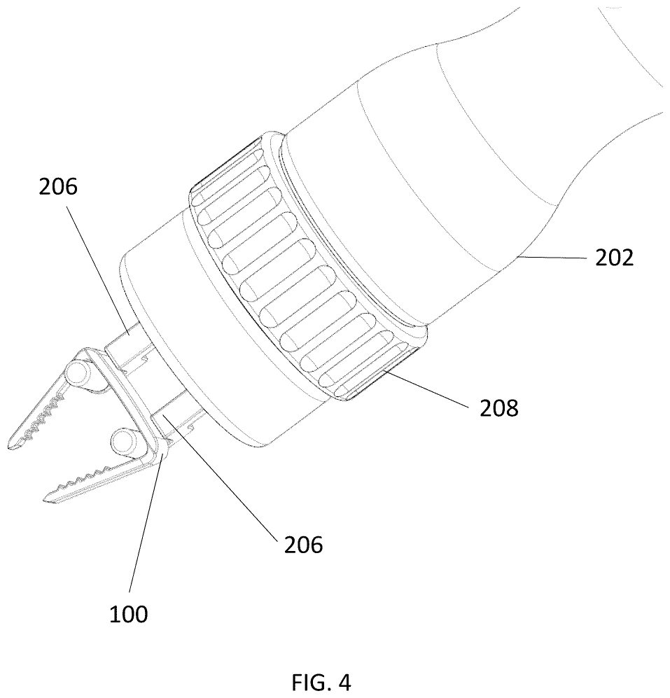

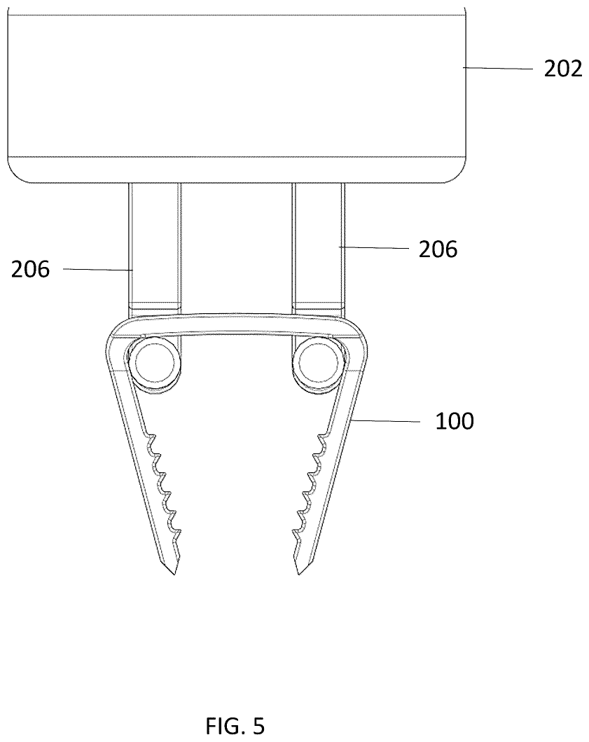

As shown in FIGS. 3-5, handle 202 has prongs 206 that would either be fixed internally or detachable. Prongs 206 may be disposed underneath bridge 104 of staple 100, for example. Handle 202 has a mechanism 208, which may be in the form of a dial 208, that when actuated or rotated prongs 206 would either distract open or compress closed. When distracting open, prongs 206 press against legs 102 of staple 100 and distract legs 102 open, resulting in legs 102 being parallel to one another.

Once legs 102 of staple 100 are distracted, ultrasonic device 200 can be turned on to convert electrical current into high-frequency mechanical vibrations. These vibrations will translate from internal components of handle 202 to prongs 206, and from prongs 206 to staple 100. The vibrations can either be a linear stroke pattern in an up-down or left-right motion, or an elliptical stroke pattern.

The high-frequency vibrations may be sustained at 20 kHz or higher. Due to the speed of the vibrations, sharp tips 108 of staple 100 would act as a cutting tool and cut a path through the bone when pressed against it. Once legs 102 are at an appropriate depth into bone, ultrasonic device 200 would be turned off, prongs 206 would be compressed closed, handle 202 would be removed from staple 100 and the bottom of bridge 104 would be tamped flush to bone.

This procedure simplifies insertion of a staple to a bone. Since the tips of the staple legs can cut their own path by use of ultrasonic technology, there is no longer a need for a drill guide or drill bits because the step of drilling the holes is removed, thus simplifying the procedure and reducing operating time.

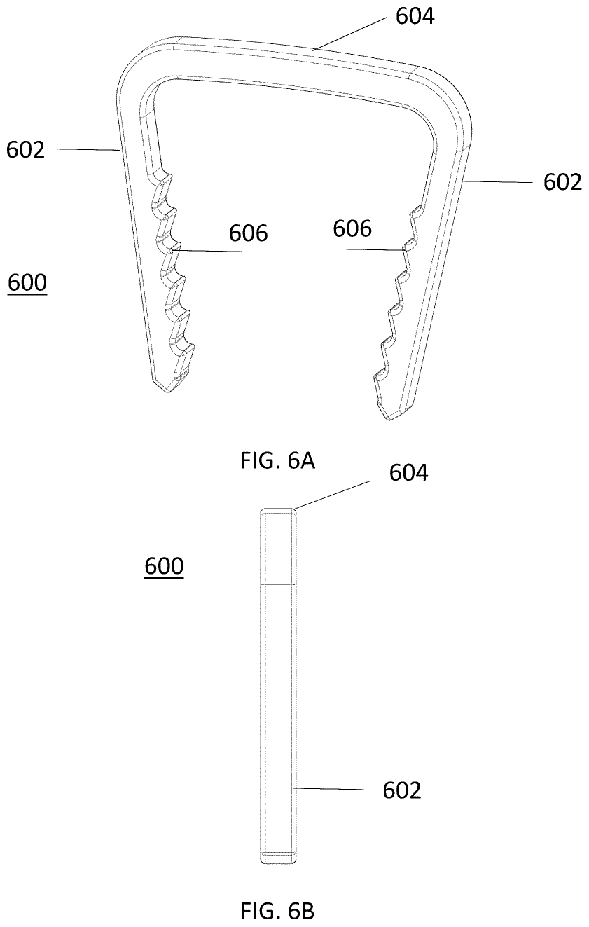

FIGS. 6A and 6B illustrate a staple 600 consistent with the principles of the present disclosure. Staple 600 may be configured to be used with a forefoot of a patient needing treatment for a fracture. Staple 600 may include legs 602 and a bridge 604. Legs 602 and bridge 604 could have different lengths depending on the size of staple chosen. Bridge 604 may be configured to have a slight curve and legs 602 may be angled inward at its resting state. Legs 602 may include teeth or serrations 606. Teeth 606 may be placed on the inside of legs 602 and angled upward. This aids in preventing staple 600 from backing out of the bone once implanted. The number of teeth or serrations 606 can vary based on the length of legs 602. Legs 602 may have tapered tips 608 which are used to help facilitate insertion of staple 600 into bone. In FIG. 6B, a side view of staple 600 illustrates bridge 604 and leg 602 thicknesses are uniform resulting in a flat staple shape.

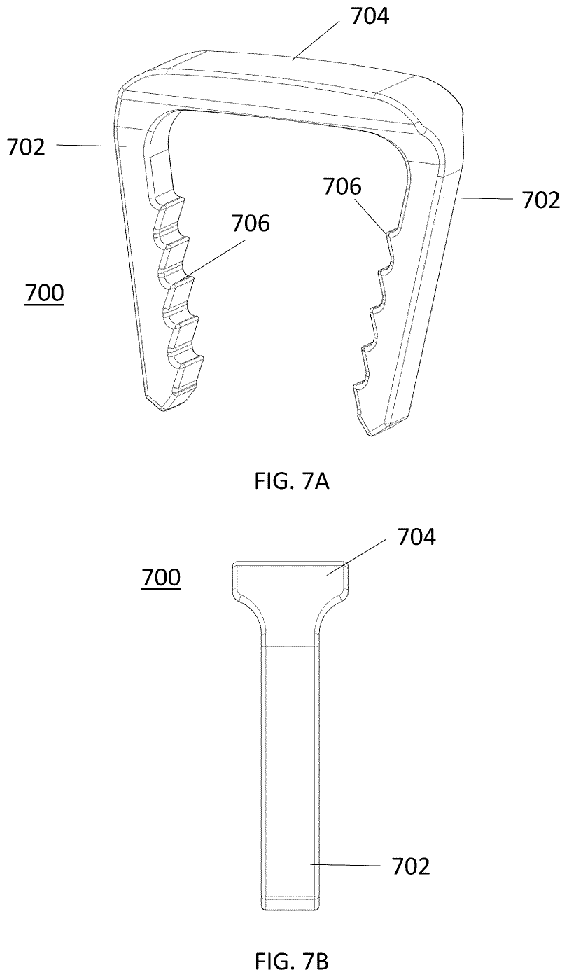

FIGS. 7A and 7B illustrate a staple 700 consistent with the principles of the present disclosure. Staple 700 may be configured to be used with a hindfoot of a patient needing treatment for a fracture. Staple 700 may include legs 702 and a bridge 704. Teeth or serrations 706 and tapered tips 708 may also be present on staple 700. As shown in FIG. 7B, a side view of staple 700 illustrates that staple 700 may be thicker than staple 600 used for the forefoot. The thickness of bridge 704 ramps up to be thicker than legs 702 in order to provide a larger compressive force that is required to hold up against the forces experienced in the hindfoot since most of the compression comes from bridge 704 of staple 700.

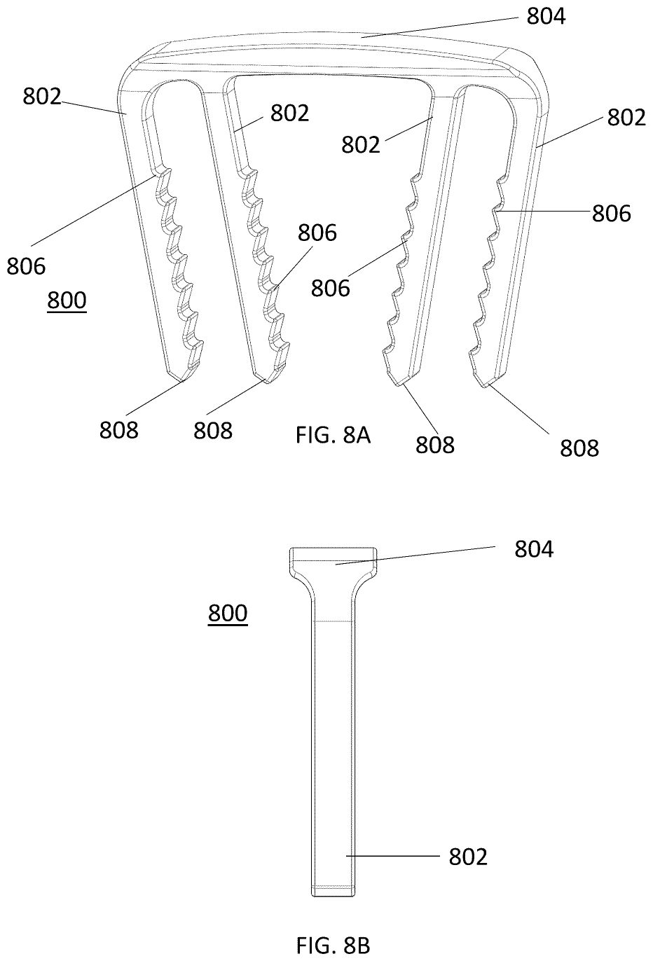

FIGS. 8A and 8B illustrate a staple 800 consistent with the principles of the present disclosure. Staple 800 may be configured to be used with a hindfoot of a patient needing treatment for a fracture. Staple 800 may have legs 802 and a bridge 804. Staple 800 may also have teeth 806 and tapered tips 808. Staple 800 may differ from previously described staples by having four legs 802 instead of two legs to provide more points of fixation and provide greater rotational stability. Staple 800 may have a thicker bridge 804 compared to legs 802 to account for the forces in the hindfoot where this staple would primarily be used. The 4 legs in this staple remain in-line to keep the narrow profile of these staples for locations that do not have a wide bone surface.

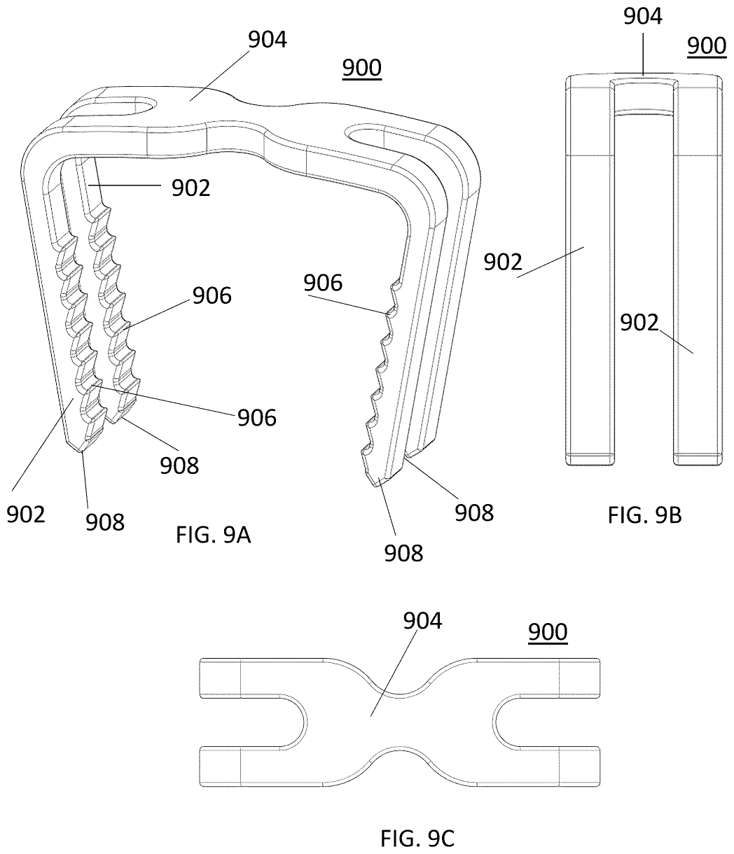

FIGS. 9A, 9B, and 9C illustrate a staple 900 consistent with the principles of the present disclosure. Staple 900 may be configured to be used with a hindfoot of a patient needing treatment for a fracture. Staple 900 may have legs 902 and a bridge 904. Staple 900 may also have teeth 906 and tapered tips 908. Staple 900 may be X-shaped when viewed from the top of bridge 904. Staple 900 provides a wider area of contact about the joint while still providing a greater amount of rotational stability. Staple 900 may have two rows of legs 902. Staple 900 may be used in areas that have enough bone surface to accommodate the overall width.

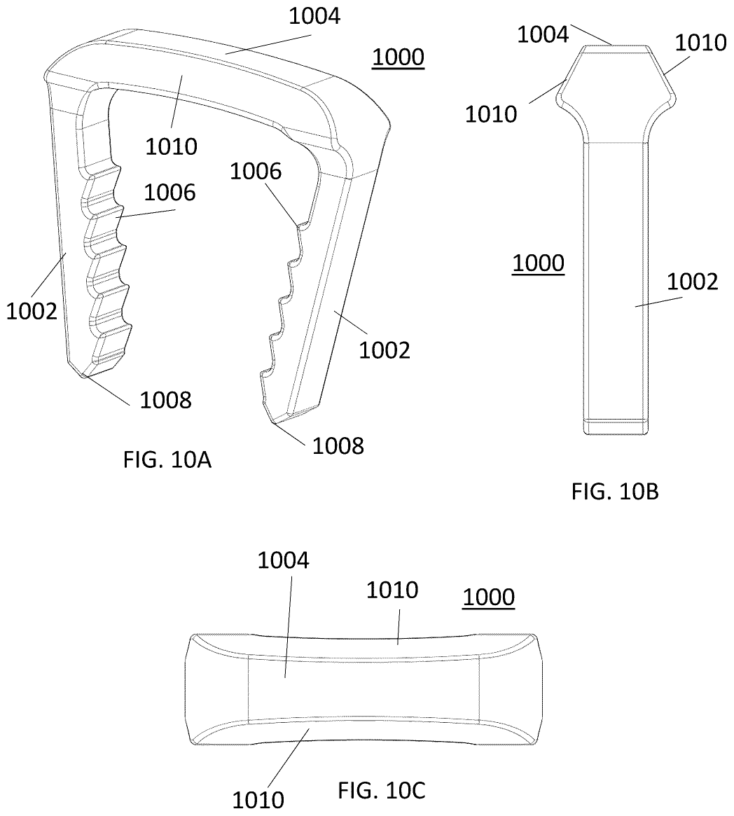

FIGS. 10A, 10B, and 10C illustrate a staple 1000 consistent with the principles of the present disclosure. Staple 1000 may be configured to be used with a hindfoot of a patient needing treatment for a fracture. Staple 1000 includes legs 1002 and a bridge 1004. Staple 1000 may also have teeth 1006 and tapered tips 1008.

As shown in FIG. 10B, a side view of staple 1000 illustrates that this staple is thicker than the forefoot style staple, such as staple 600. The thickness of bridge 1004 ramps up to be thicker than legs 1002 in order to provide a larger compressive force that is required to hold up against the forces experienced in the hindfoot since most of the compression comes from the bridge of the staple. There are angled cuts 1010 on the top portion of bridge 1004. As shown in FIG. 10C, a top view of staple 1000 shows that angled cuts 1010 of bridge 1004 allow bridge 1004 to thin out slightly towards the center to facilitate bending at this location when engaging the staple with an inserter to implant into bone. Bridge 1004 contains angled cuts 1010 such that a width of a top portion of the bridge is less than a width of a bottom portion of bridge 1004. During implantation into bone, a force is applied to bridge 1004 and legs 1002 splay open such that tapered tips 1008 are a greater distance apart than an original position. After staple 1000 is implanted, legs 1002 move toward each other to provide a compressive force to the site of the fracture.

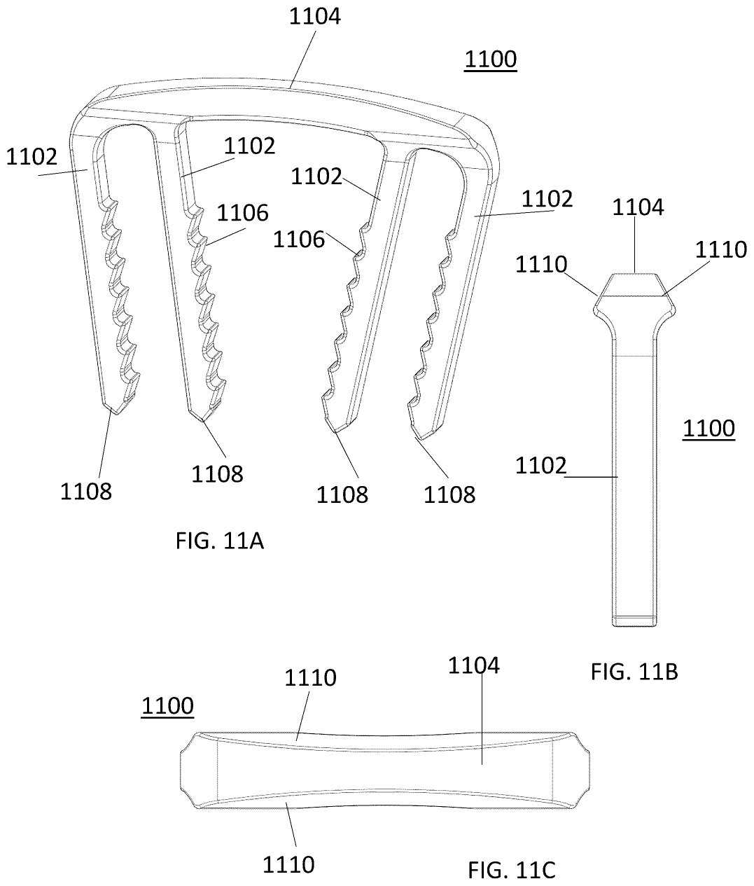

FIGS. 11A, 11B, and 11C illustrate a staple 1100 consistent with the principles of the present disclosure. Staple 1100 may be configured to be used with a hindfoot of a patient needing treatment for a fracture. Staple 1100 may have legs 1102 and a bridge 1104. Staple 1100 may also have teeth 1106 and tapered tips 1108. Staple 1100 may have four legs 1102 instead of two legs to provide more points of fixation and provide greater rotational stability. Staple 1100 may have a thicker bridge 1104 compared to legs 802 to account for the forces in the hindfoot where this staple would primarily be used. The 4 legs in this staple remain in-line to keep the narrow profile of these staples for locations that do not have a wide bone surface. Staple 1100 may also have angled cuts 1110 on the top of bridge 1104 as shown in the side and top views of FIGS. 11B and 11C, respectively.

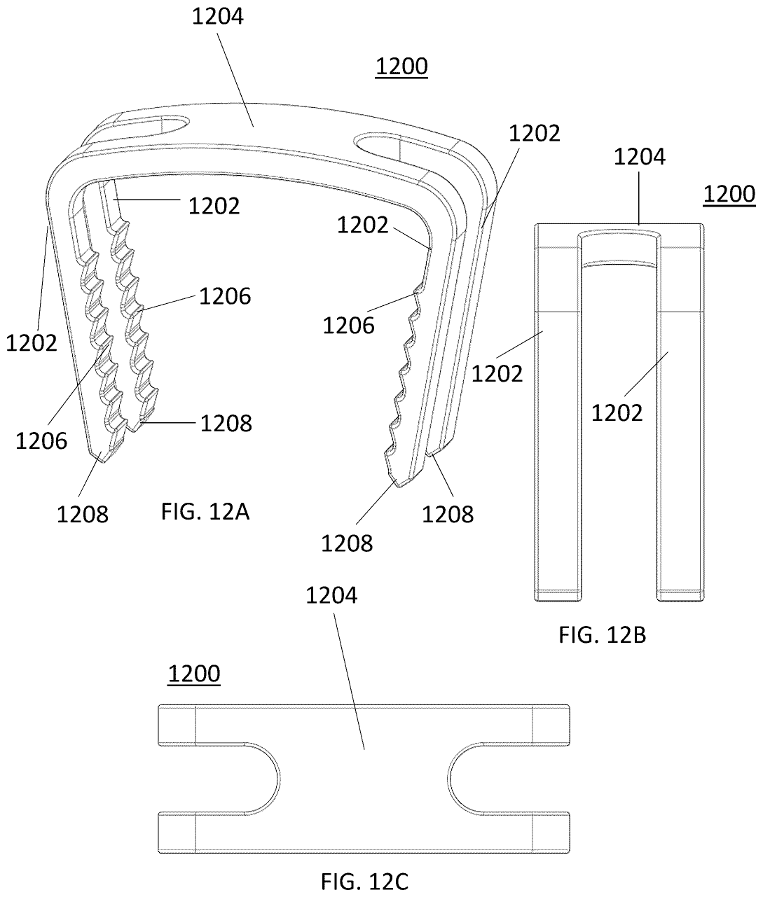

FIGS. 12A, 12B, and 12C illustrate a staple 1200 consistent with the principles of the present disclosure. Staple 1200 may be configured to be used with a hindfoot of a patient needing treatment for a fracture. Staple 1200 may have legs 1202 and a bridge 1204. Staple 1200 may also have teeth 1206 and tapered tips 1208. Staple 1200 may be I-shaped when viewed from the top of bridge 1204. Staple 1200 provides a wider area of contact about the joint while still providing a greater amount of rotational stability. Staple 1200 may have two rows of legs 1202. Staple 1200 may be used in areas that have enough bone surface to accommodate the overall width.

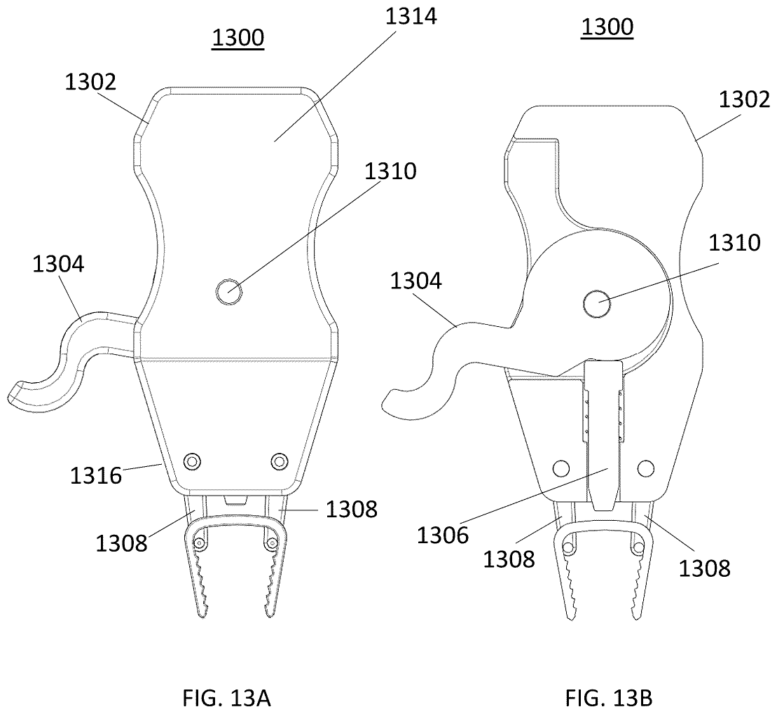

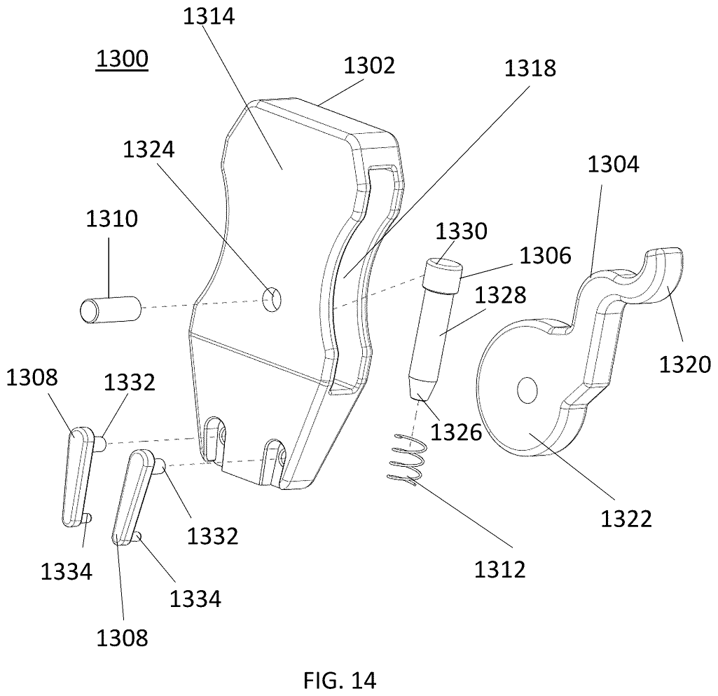

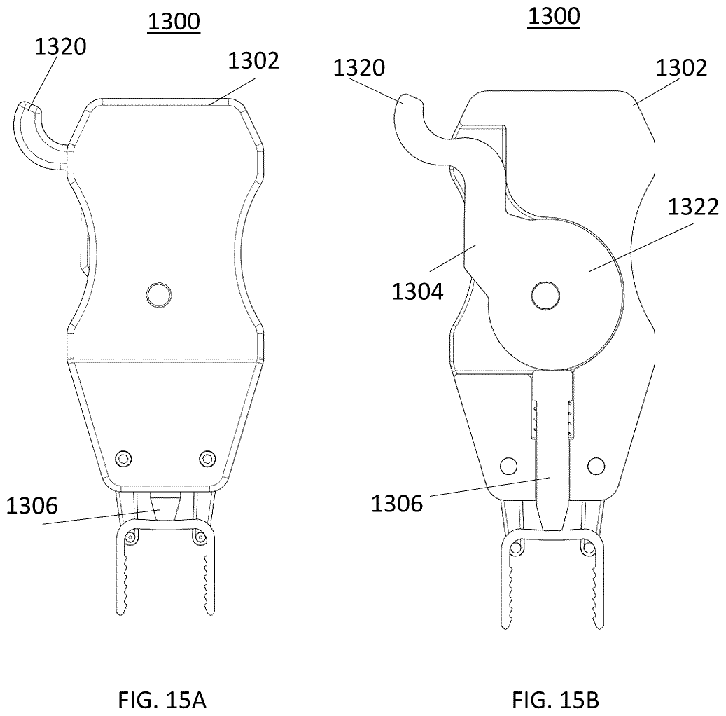

FIGS. 13A-18 illustrate an inserter 1300 consistent with the principles of the present disclosure. Inserter 1300 includes a housing 1302, a cam lever 1304, a post 1306, tips 1308, a center pin 1310, and a spring 1312. In operation, a staple is held by tips 1308 as shown in FIGS. 13A and 13B with cam lever 1304 in a first position. When cam lever 1304 is in a second position as shown in FIGS. 15A and 15B, post 1306 is moved by cam lever 1304 to provide a force on the bridge of the staple to splay the legs wider than their original position and the staple is inserted into the bone. Components of inserter 1300 are described in further detail below.

Housing 1302 contains the other components of inserter 1300 and provides the surgeon with flat outer surfaces 1314 to grip when inserting a nitinol staple into bone. A distal end 1316 of inserter 1300 will decrease in width to limit how much housing 1302 occludes the view of the staple being inserted into the surgical site. Housing 1302 includes multiple holes for pins and posts to either hold components together or interact and deploy the staple. Housing 1302 also includes a pocket 1318 from one side that allows for the deployment mechanism to be assembled.

Cam lever 1304 has an arm 1320 that is offset from a cam 1322 and extends beyond the profile of housing 1302 when assembled. Cam lever 1304 will have a pin hole 1324 that is located at the center of cam 1322 that cam 1322 will rotate about when assembled. Cam 1322 itself will be designed such that along its circumference, the radius will change depending on where it is in contact with the mating components.

Post 1306 has a distal end 1326 that will extend out of distal end 1316 of housing 1302 when assembled. Post 1306 has a shaft 1328 and a proximal end 1330 that will contact cam lever 1304 during use. Shaft 1328 may have a constant diameter. Distal end 1326 will be tapered to avoid interfering with tips 1308 during assembly and use. Distal end 1326 of post 1306 pushes down on the staple bridge. Proximal end 1330 of post 1306 has a larger diameter than shaft 1328. This provides a greater surface area for the cam lever 1304 to slide against. This also allows for spring 1312 to push against post 1306 and helps to retain it inside housing 1302.

Tips 1308 will be press fit into housing 1302 during assembly. Each of tips 1308 may have two bosses 1332 and 1334, one on each end of the part. Boss 1332 on a proximal end will be what is press fit into housing 1302 and boss 1334 on a distal end will be used to load the staple onto the staple inserter. The staple will rest on boss 1334 on the distal end of tip 1308. Tips 1308 will be press fit into a staple inserter so the staple will rest on two bosses 1334.

Center pin 1310 may be press fit into one side of housing 1302, go through pin hole 1324 of cam lever 1304 and then press fit again through the other side of housing 1302. Center pin 1310 holds cam lever 1304 in place while allowing it to rotate around it.

Spring 1312 may be placed around shaft 1328 of post 1306 and will fit into pocket 1318 in housing 1302. Spring 1312 will push post 1306 into housing 1302 to keep the tip of post 1306 out of the way of the staple for easy assembly.

To load the staple onto inserter 1300, cam lever 1304 is pulled down to its unlocked position as shown in FIGS. 13A and 13B. Cam 1322 on cam lever 1304 is positioned so that the smallest radius is facing distally and resting against post 1306. This allows post 1306 more room to insert into housing 1302. This creates room for the bridge of the staple to seat onto distal bosses 1334 of tips 1308.

Once the staple is in place, cam lever 1304 is actuated up into its locked position as shown in FIGS. 15A and 15B. This will cause cam 1322 on cam lever 1304 to rotate such that the largest radius is facing distally and this will slide against post 1306 and push it down. Post 1306 will continue to be forced out of housing 1302 until it touches the bridge of the staple and forces the bridge of the staple to bend. While the staple bridge is bending, the legs of the staple will become parallel. This is the locked, deployed position that allows the staple to be inserted into bone.

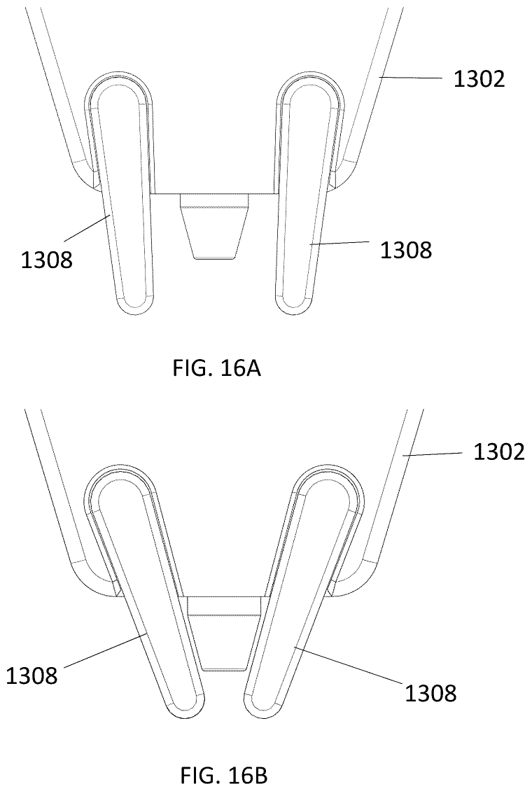

Once the staple is inserted into bone, the surgeon would pull cam lever 1304 down to release post 1306 from pressing down on the staple and then slide tips 1308 out from under the staple bridge. One of the surfaces of the inserter 1300 could be used to tamp the staple down flush with the bone or an additional tool could be used to tamp. The grooves within housing 1302 that tips 1308 get inserted to are what determine the distance and angle between distal bosses 1334 of tips 1308. The angle of the groove can be changed depending on the bridge length of the staple that is being attached. Each staple bridge length will have a separate staple inserter with a set tip angle. FIGS. 16A and 16B illustrate inserters with different tip angles.

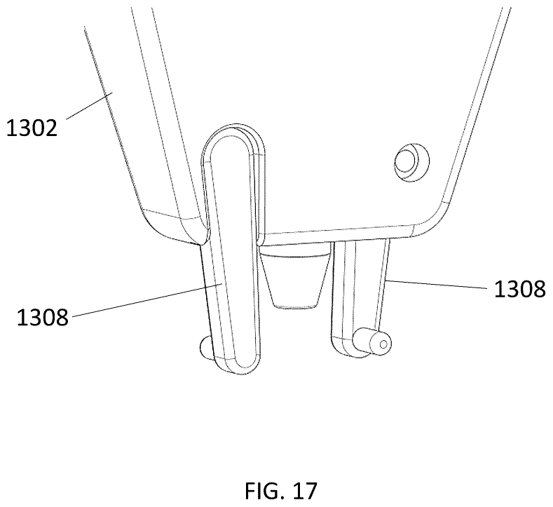

As shown in FIG. 17, tips 1308 could be assembled on opposite sides of housing 1302. This would require a surgeon to twist counter-clockwise to remove tips 1308 out from under the bridge of the staple as opposed to sliding it.

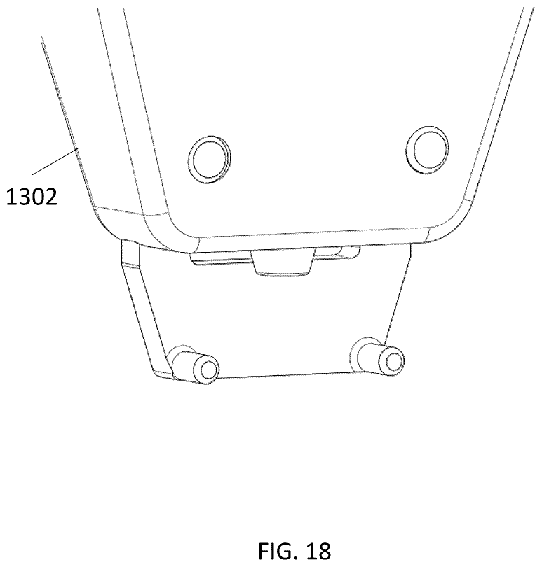

As shown in FIG. 18, tips 1308 could be implemented as a single component that are bridged together with more material. This would reduce the overall components needed and case the assembly method.

Inserter 1300 allows for a quick motion of the cam lever 1304 to release the staple after it has been inserted. Inserter 1300 also has the ability to be reloaded onto a staple if the surgeon decides they need to move it or take it out completely after implantation. Some existing staple inserters do not allow for re-insertion of the staple inserter once the staple has been removed.

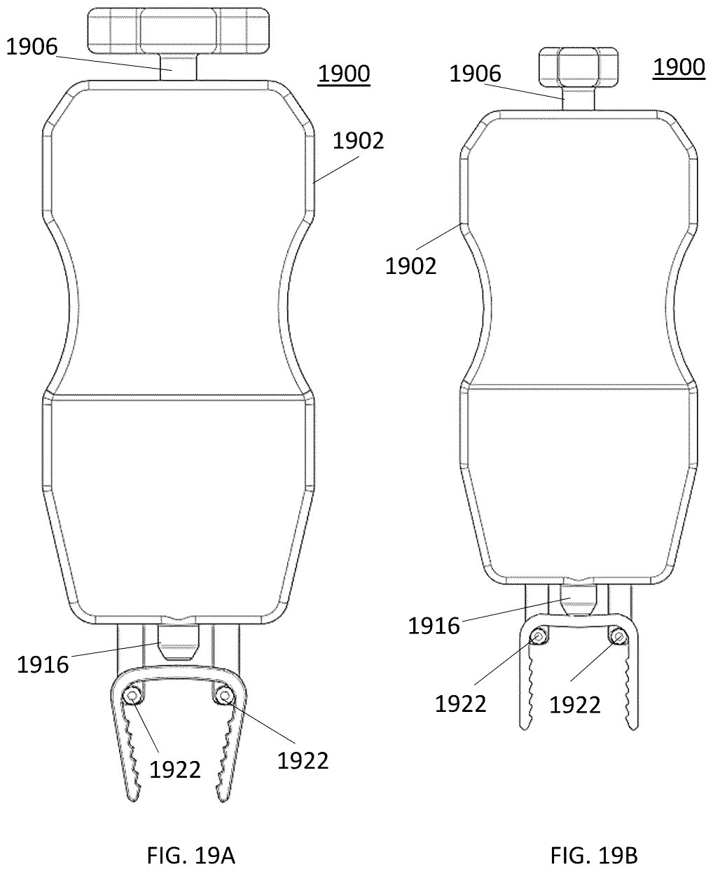

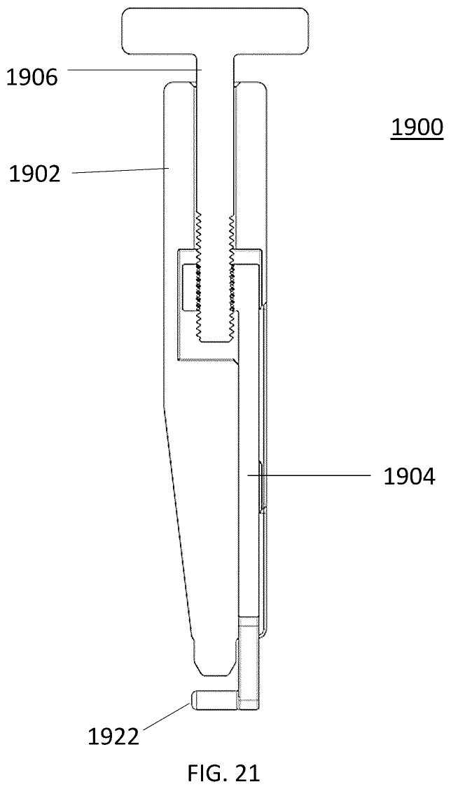

Turning now to FIGS. 19A-21, an inserter 1900 is illustrated that is consistent with the principles of the present disclosure. Inserter 1900 includes a housing 1902, an arm 1904, and a threaded post 1906. In operation, a staple is loaded on to arms 1904 and as the threaded post 1906 is rotated, arm 1904 are retracted towards a proximal end of housing 1902 and a bridge of the staple engages a portion of housing 1902 to bend the bridge and splay the legs of the staple further apart. Each of the components of inserter 1900 will be described in further detail below.

Housing 1902 may contain the other components of the inserter 1900 and provide the surgeon with flat outer surfaces to grip when inserting a nitinol staple into bone. A distal end 1908 of housing 1902 will decrease in width to limit how much housing 1902 occludes the view of the staple being inserted into the surgical site. Housing 1902 includes a hole 1910 on a proximal end 1912 for the threaded post 1906 to be placed in during assembly. Housing 1902 also includes a pocket 1914 on the back side that allows for arm 1904 to be placed in during assembly and also to translate during use. Housing 1902 includes a tip or pin 1916 that protrudes down from distal end 1908 which will interact with the bridge of the staple during use.

Arm 1904 has a threaded hole 1918 on a proximal end 1920 of arm 1904 to mate with the threaded post 1906. Arm 1904 has two bosses 1922 on a distal end 1924 of arm 1904 that is used to load the staple onto inserter 1900. The staple will rest on both bosses 1922 while being loaded.

Threaded post 1906 will have a distal end 1926 that is threaded to interact with arm 1904. A proximal end 1928 of threaded post 1906 will have a wing nut shape that will stick out past proximal end 1912 of housing 1902 for the surgeon to rotate clock-wise to load a staple and counter clock-wise to remove a staple.

Inserter 1900 is designed to push down on the top of the middle bridge of a staple while also holding onto the underside of the bridge near the corners. This causes the bridge of the staple to bend downwards which allows the legs of the staple to flex outward causing them to become parallel to each other for insertion into bone. For assembly, arm 1904 is placed into housing 1902 from the back side and threaded post 1906 is inserted into hole 1910 in housing 1902 from proximal end 1912 and threaded into threaded hole 1918 on arm 1904.

To load the staple onto inserter 1900, threaded post 1906 is rotated counter clock-wise which will translate arm 1904 distally. This will create enough space between bosses 1922 on arm 1904 and tip 1916 of housing 1902 as shown in FIG. 19A. With the space created, the staple can be seated onto distal bosses 1922 of arm 1904.

Once the staple is in place, threaded post 1906 is rotated clock-wise. This will cause arm 1904 to translate proximally back into housing 1902. Tip 1916 on housing 1902 will come in contact with the bridge of the staple as threaded post 1906 is rotating and will force the bridge of the staple to bend. While the staple bridge is bending, the legs will become parallel. This is the locked, deployed position that allows the staple to be inserted into bone as shown in FIG. 19B.

Once the staple is inserted into bone, the surgeon would rotate threaded post 1906 counter clock-wise to release the tension created on the staple and allow the legs to bend inward and then slide bosses 1922 of arm 1904 out from under the staple bridge. One of the surfaces of inserter 1900 could be used to tamp the staple down flush with the bone or an additional tool could be used to tamp. The distance between bosses 1922 of arm 1904 can be changed depending on the bridge length of the staple that is being attached. Each staple bridge length will have a separate staple inserter with a set distance between the bosses.

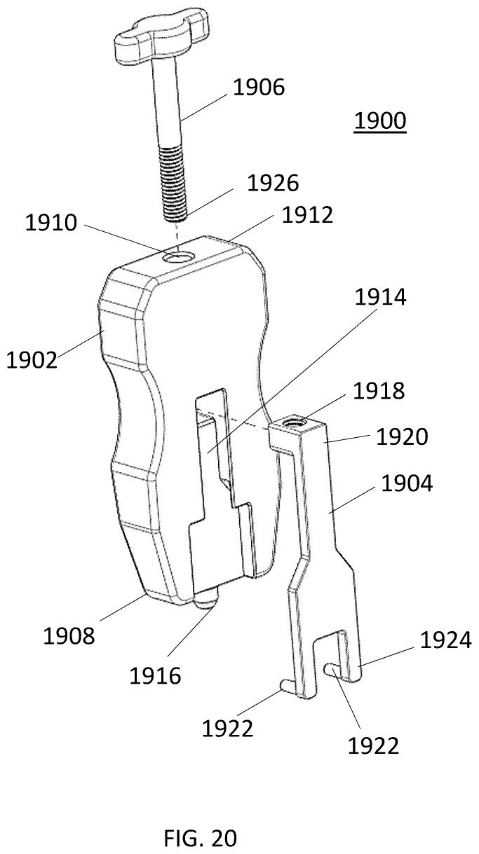

Arm 1904 may be embedded into housing 1902 to prevent disassembly. FIG. 20 is an exploded view of inserter 1900 and shows how arm 1904 fits within pocket 1914 of housing 1902 and allows it to still translate for deploying and removing from the staple.

Inserter 1900 allows for a threaded mechanism to release the staple after it has been inserted. Inserter 1900 also has the ability to be reloaded onto a staple if the surgeon decides they need to move it or take it out completely after implantation. Some existing staple inserters do not allow for re-insertion of the staple inserter once the staple has been removed.

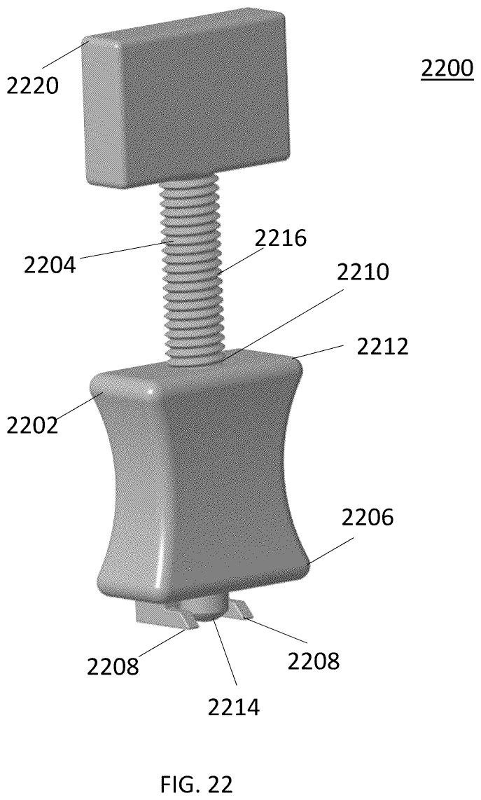

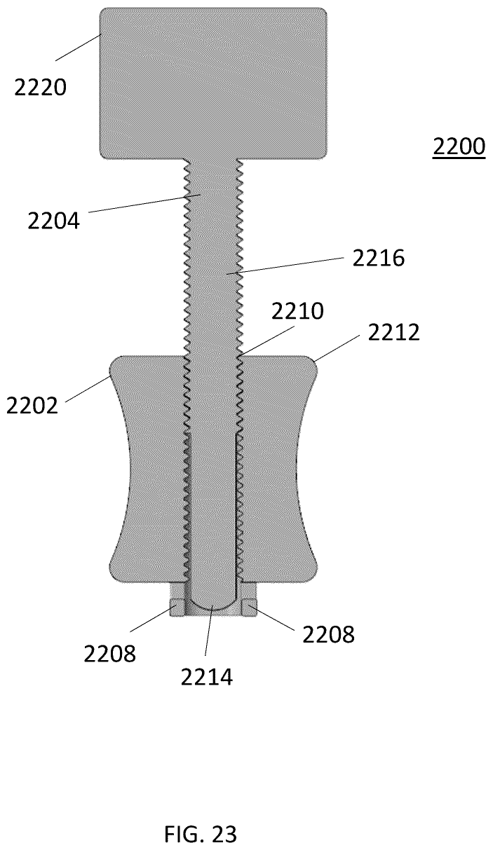

FIGS. 22 and 23 illustrate an inserter 2200 consistent with the principles of the present disclosure. Inserter 2200 includes a housing 2202 and a threaded post 2204. These components are described in further detail below.

Housing 2202 may contain the other components of inserter 2200 and provide the surgeon with flat outer surfaces to grip when inserting a nitinol staple into bone. A distal end 2206 of inserter 2200 includes prongs 2208 that act as a resting surface for the staple. Housing 2200 includes a threaded thru hole 2210 on a proximal end 2212 for threaded post 2204 to be placed in during assembly.

Threaded post 2204 has a distal end 2214 that is smooth and rounded to interact with the bridge of the staple. Threaded post 2204 contains a shaft 2216. A middle section 2218 of shaft 2216 will be threaded to interact with housing 2202. A proximal end 2220 of post 2204 may have a wing nut shape that will stick out past proximal end 2212 of housing 2202 for the surgeon to rotate clock-wise to load a staple and counter clock-wise to remove a staple.

Inserter 2200 is designed to push down on the top of the middle bridge of a staple while also holding onto the underside of the bridge near the corners. This causes the bridge of the staple to bend downwards which allows the legs to flex outward causing them to become parallel to each other for insertion into bone. For assembly, the threaded post 2204 is threaded into housing 2202 from proximal end 2212 of housing 2202.

To load the staple onto inserter 2200, threaded post 2204 is rotated counter clock-wise which will translate a tip of threaded post 2204 distally. This will create enough space between prongs 2208 on housing 2202 and the tip of threaded post 2204. With the space created, the staple can be seated onto prongs 2208 of housing 2202.

Once the staple is in place, threaded post 2204 is rotated clock-wise. This will cause threaded post 2204 to translate distally. The tip of threaded post 2204 comes in contact with the bridge of the staple as threaded post 2204 is rotating and will force the bridge of the staple to bend. While the staple bridge is bending, the legs will become parallel. This is the opened, deployed position that allows the staple to be inserted into bone.

Once the staple is inserted into bone, the surgeon would rotate threaded post 2204 counter clock-wise to release the tension created on the staple and allow the legs to bend inward and then slide prongs 2208 of housing 2202 out from under the staple bridge. One of the surfaces of inserter 2200 could be used to tamp the staple down flush with the bone or an additional tool could be used to tamp. The distance between prongs 2208 of housing 2202 can be changed depending on the bridge length of the staple that is being attached. Each staple bridge length will have a separate staple inserter with a set distance between prongs.

Inserter 2200 allows for a threaded mechanism to release the staple after it has been inserted. Inserter 2200 has the ability to be reloaded onto a staple if the surgeon decides they need to move it or take it out completely after implantation. Some existing staple inserters do not allow for re-insertion of the staple inserter once the staple has been removed. Inserter 2200 is low-profile and allow for visualization of the surgical site that the staple is being inserted into.

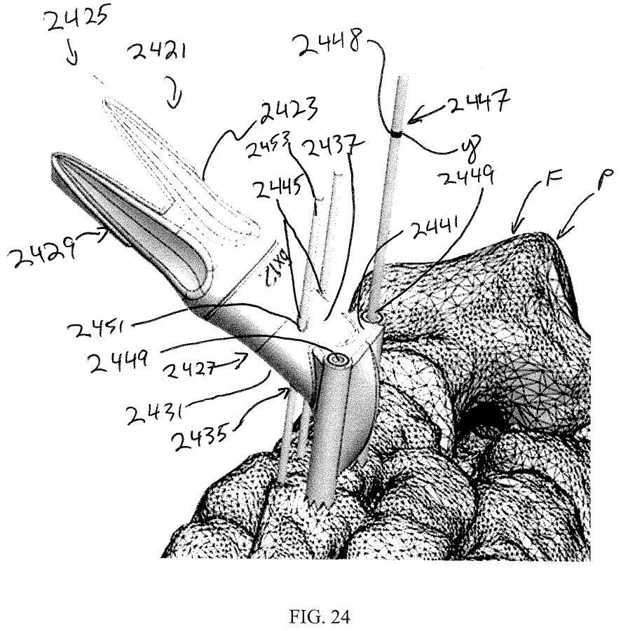

The staples and staple inserters of this disclosure may comprise components, subassemblies, or devices of any number of a variety of surgical-staple implantation systems for use in bone fracture repair procedures generally, or foot fracture procedures more particularly. Additional components, subassemblies, or devices, and their associated features, are disclosed hereinbelow with reference to FIGS. 24-33.

Referring now to FIGS. 24-29, a hand-held drill guide 2421 may be used as part of a surgical-staple implantation system. Drill guide 2421 may be used in performing certain aspects of related procedures on a patient P, a portion of whose foot F is shown in FIG. 24, being acted upon by drill guide 2421. In general terms, drill guide 2421 includes structures and features further discussed below to assist a medical practitioner or other user in drilling pilot holes on either side of a fracture in foot F with a surgical drill bit 2447. The user's hand holds drill guide 2421 by grasping surface 2429 thereof, and, in the illustrated procedure, fixes the location and alignment of the operative, distal end of drill guide 2421. This is done by pinning k-wires 2453 at respective parallel locations offset from where drill bit 2447 will be operating.

To the foregoing ends, grasping surface 2429 extends between opposite proximal and distal ends 2425, 2427 of handle 2423 of the drill guide 2421. For purposes of clarity regarding use of the terms “proximal” and “distal” for the components illustrated in FIGS. 24-33, such terms shall have their ordinary meanings to one of skill in the art. For example, “proximal” when referring to portions of component disclosed herein describes location or locations which are generally closer to the medical practitioner when such component is in use on a patient, whereas “distal” under such circumstances refers to portions generally further from the practitioner when in use on a patient, and thus closer to, or in operative proximity to, such patient.

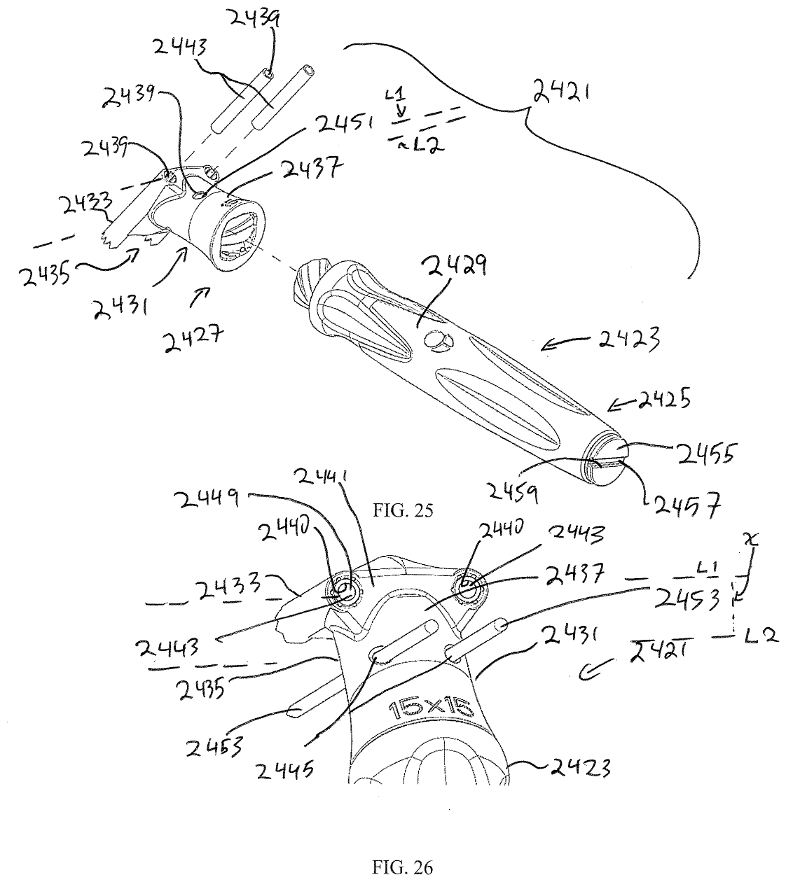

Drill guide 2421 includes a head 2431, which may be selectively attached or detached to handle 2423 by any suitable means. In the illustrated implementation, as shown in FIG. 25, opposing mating portions of handle 2423 and head 2431 may be engaged or disengaged to attach or detach the components from each other. As shown, a recess or pocket extends into a surface of head 2431 opposite a corresponding protrusion from a surface of handle 2423 opposite head 2431. The recess and protrusion combination may involve threads or other securement mechanisms. Still other arrangements and structures for removably attaching head 2431 to handle 2423 are likewise suitable.

Head 2431 has a head surface or head surfaces which, when secured to handle 2423, extend distally to define a drill guide tip 2433. One head surface includes an engagement surface 2435 which is located on head 2431 so that such engagement surface may be brought into operative proximity to a fracture of patient P to be treated. In the illustrated implementation, another head surface includes a user-accessible surface 2437. User-accessible surface is, as its name suggests, able to be accessed by the medical practitioner when engagement surface 2435 is positioned in operative proximity to the fracture to be treated.

Head 2431 has portions which define multiple bores 2439, which bores extend between the two head surfaces described above. A first set 2443 of the bores are sized to slidably and rotatably receive the surgical drill bit 2447 described previously and shown in FIG. 24. The inner surfaces of the bores of first set 2443 have a hardness which is greater than the hardness of drill bit 2447 to protect head 2431 from damage when drill bit 2447 is advanced and rotated within the bores, especially the portions of head 2431 surrounding the bores of first set 2443. One suitable way to provide for such inner surfaces having hardness greater than drill bit 2447 is shown in the illustrated embodiment as tubes 2448 having the requisite harder or hardened inner surfaces, and which are secured by interference fit or other suitable means at the distal, drill guide tip portion of head 2431. Hardened inner surfaces of first set 2443 of the bores may be accomplished by any number of other chemical, metallurgical, molding, casting or similar processes, with hardened inner surfaces being removable relative to head 2431 or fused, connected or otherwise integrated.

The bores of first set 2443 are located at drill guide tip 2433 in laterally spaced relation to each relative to a central longitudinal axis of drill guide 2421, along a first lateral axis L1. Given these bores are sized to receive a suitable drill bit associated with the procedure therein, bores 2439 of first set 2443 define drill guide holes 2449.

A second set of bores 2445 is spaced longitudinally in the proximal direction from the drill guide holes and define k-wire holes 2451. K-wire holes 2451 are likewise laterally spaced in relation to each other about the same central longitudinal axis of drill guide 2421, but are located along a second, lateral axis L2 of head 2431. The second lateral axis L2 is disposed parallel to the first lateral axis L1, thereby defining an offset distance x from drill guide holes 2449. K-wire holes 2451 are sized to slidably and rotatably receive k-wires 2453 therethrough.

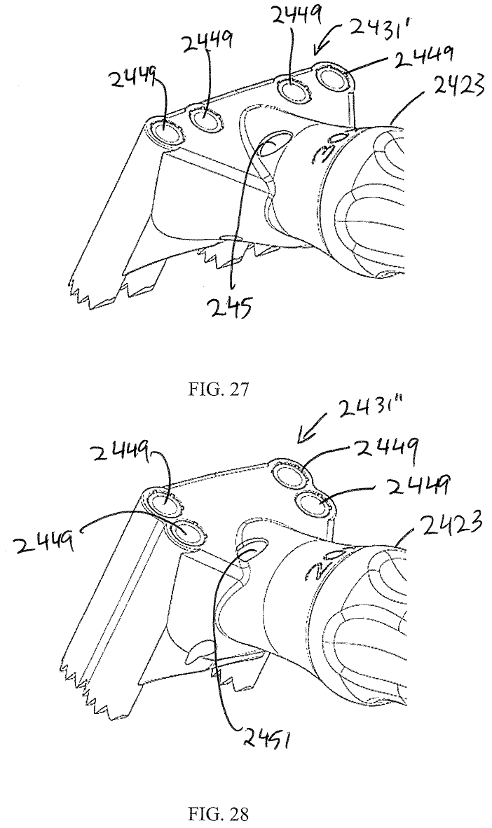

Drill guide 2421 may be associated, through a kit, as part of a multi-component surgical staple implantation system, or otherwise, with multiple heads 2431. The multiple heads 2431 may be identical for redundancy, but may likewise have varying dimensions, bore configurations, bore quantities, bore-spacings, and other parameters so that drill guide 2421 finds multiple applications for multiple surgical staple procedures, using any of a variety of surgical staples. In the illustrated implementation, head 2431 in FIGS. 24-26 may be used to form two pilot holes corresponding to use with any of a variety of two-leg surgical staples. Referring now to FIG. 27, head 2431′ has defined therein four of the drill guide holes 2449 laterally disposed so as to be useful with suitable four-leg, in-line surgical staples. Still further, referring to FIG. 28, head 2431″ has four drill guide holes 2449 defined therein, but in this case, the holes are both laterally and longitudinally spaced to correspond to a four-leg, I-beam staple.

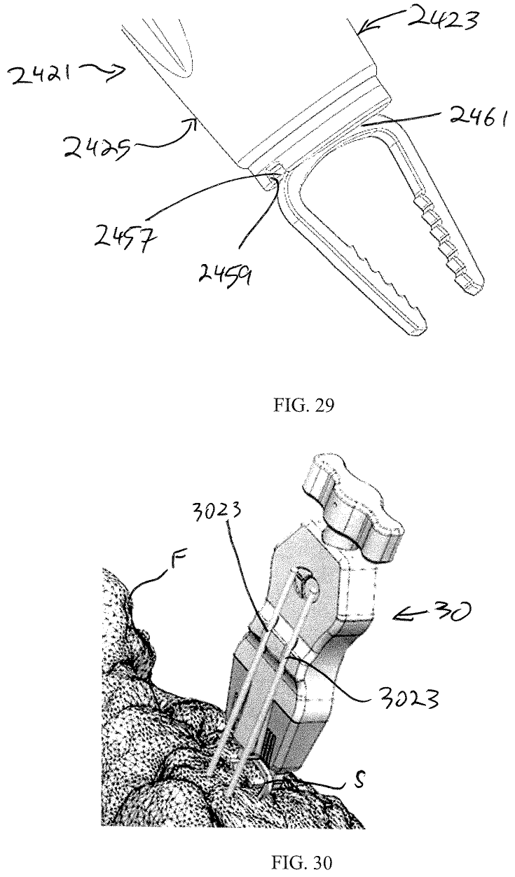

Referring more particularly now to FIGS. 25 and 29, drill guide 2421 may include a tamp 2455 located at proximal end 2425 of handle 2423. Tamp 2455 may be selectively employed to further seat or otherwise advance surgical staples after implantation in bone in various surgical staple procedures. Tamp 2455 has portions defining a slot 2457 therein, which slot has walls 2459 dimensioned to receive bridge portion 24651 of a corresponding staple as shown in FIG. 29. Tamp 2455 and its slot 2457, in the illustrated embodiment, are connected to or integrated with handle 2423, but tamp 2455 may be selectively attachable and detachable to drill guide 2421 at any number of locations or may be an independent component of a surgical staple implantation system. In the illustrated implementation, slot 2457 is located relative to grasping surface 2429 of handle 2423 so that, if slot 2457 receives bridge 2461 of a staple after insertion into a bone of patient P, distally directed force, whether manual or mechanically applied to tamp 2455 is transmitted to the bridge portion 2461 and its corresponding staple to achieve implantation objectives.

Hand-held drill guide 2421 may be used in any number of surgical staple procedures and in conjunction with a variety of surgical staple implantation systems and their components. In one suitable method of implanting a surgical staple in a bone-fracture-repair procedure, drill guide 2421 may be used to drill pilot holes once an implantation location and a suitable staple configuration has been determined. A drill guide head 2431 suitable for the selected staple and its leg configuration is attached to drill guide 2421 prior to positioning guide 2421. Thereafter, drill guide head 2431 is brought into operative proximity to the implantation location, as shown in one possible exemplary location shown in FIG. 24. As such, drill guide holes 2449 have been positioned as appropriate at the implantation location, such as with a staple leg or legs on either side of the fracture which is the subject of the procedure.

In one possible series of steps for this procedure, once the drill guide head 2431 has been suitably positioned, the location of the corresponding drill guide holes 2449 can be pinned, that is, secured or fixed, to improve accuracy of pilot hole drilling, by using k-wires 2453. In one possible approach, before drilling pilot holes, k-wires 2453 are suitable advanced through k-wire holes 2451 by any means suitable for distal ends of k-wires to engage bone or tissue underlying k-wire holes 2451. Once the drill guide holes have been pinned using k-wires in this manner, surgical drill bit 2447 may be advanced through each of the drill guide holes to form the requisite pilot holes for the staple to be subsequently implanted.

Such procedures may include still other steps to optimize pilot hole creation. For example, the surgical implantation system may employ drill bit 2447 with a suitable mark 2448 at a longitudinal location y thereon corresponding to a limit depth of the pilot hold or associated with the staple configuration being used. A suitable component of the surgical implantation system is configured to detect when such mark 2448 has been advanced to a predetermined position or by a predetermined amount corresponding to the limit depth. Upon such detection, any of a variety of signals are given to cease advancing of the drill bit relative to the bone or implantation location. In the event braces, jigs, or other mechanisms are associated with drill bit 2447, suitable mechanical means may be triggered upon such detection to mechanically arrest further advance. In another variation, a clutch or other engagement means associated with drill bit movement may stop advancement. In still other variations, an audible signal is generated and the user manually ceases advancement of the drill bit.

By virtue of k-wire holes 2451 being laterally offset from drill guide holes 2449, the advantages of redundant or improved fixation of drill guide head 2431 with k-wires may be gained without impeding drill bit operations through drill guide holes 2449.

In still further implementations and associated procedures using drill guide 2421, k-wires 2453 have diameters smaller than those of drill guide holes 2449, and thus k-wires may be employed to improve pinning of drill guide head 2421 relative to the implantation site by using the k-wires 2453 with the drill guide holes 2449. In one possible series of steps, a first one of the k-wires 2453 may be fixed between bone and the drill guide by advancing such k-wire through a first one of the drill guide holes, rather than (or in addition to) using k-wire holes 2451. Thereafter, formation of pilot holes using drill bit 2447 may be accomplished through one or more of the other drill guide holes not occupied by k-wire or k-wires 2453.

Further variations employing k-wires in drill guide holes with or without using k-wire holes, in sequence with or in parallel with pilot hole drilling operations are contemplated. So, for example, a number of variations of the foregoing would apply to procedures with drill guide head 2431′ or 2431″ of FIGS. 27 and 28, such as fixing a second one of the k-wires 2453 between the drill guide and the implantation location by advancing the second k-wire through a second one of the drill guide holes 2449.

A further variation may involve not merely using k-wires 2453 to pin drill guide head 2431 through drill guide holes 2449 but may involve advancing one or more k-wires into pilot holes drilled in preceding steps at the implantation location.

Drill guide 2421 may be used in conjunction with any of a variety of staple inserters, including those disclosed herein, with reference to FIGS. 3-5 and 13-23. Drill guide 2421 may also be advantageously used with staple inserter 3021 (FIG. 30). Staple inserter 3021 has many of the features of the staple inserters described with reference to the previous Figs., but in addition has features which permit staple inserter 3021 to be used with k-wires 2453, under suitable procedures.

In one exemplary implementation of staple inserter 3021 and its corresponding workflow, two or more slots 3023 are formed on one of the sides of staple inserter 3021 and configured to receive respective k-wires 2453 slidably therein. Slots 3023 extend longitudinally, that is, proximally and distally, over a length. In addition, slots are laterally spaced from each other by amounts corresponding to the lateral spacing of the k-wires engaged at the implantation site. Still further, the slots 3023 occupy a longitudinally extending plane that is laterally offset by the longitudinal plane occupied by a staple being held by the staple inserter 3021 for implantation (or insertion). As such, when k-wires 2453 are received in slots 3023, the k-wires 2453 act as guide wires to the slots 3023 for staple insertion operations associated with staple inserter 3021.

Suitable staple implantation procedures are apparent from the foregoing description. In one possible procedure illustrated in FIGS. 24 and 30, the pair of k-wires 2453 is fixed at the offset distance x relative to drill guide holes 2449, as well as any corresponding pilot holes, as may be necessary, depending on insertion protocols of staple inserter 3021. Drill guide 2421 may be removed from the implantation location while leaving previously affixed k-wires 2453 at their offset location relative to the implantation location.

Thereafter, slots 3023 may be used to align a staple S to be implanted with the pilot holes previously formed, because the slots 3023 are offset by the same distance x as were the k-wires 2453 relative to the drill-guide holes 2449 and any resulting pilot holes. The side of staple inserter 3021 in which slots 3023 are formed is positioned so that distal portions of k-wires 2453 are received into respective slots 3023. Thereafter, advancement of staple S into the implantation location, including any pilot holes formed therein, occurs under guiding influence of slots 3023, by application of suitable distally oriented force to the staple while the k-wires remain received in the slot. As such the staple S is implanted or inserted at the implantation location and the associated legs of the staple advance into bone, tissue or through the pilot holes.

In still other possible procedures apparent from the foregoing description, after a staple has been inserted by the staple inserter and separated therefrom, a user may wish to further advance such staple into the implantation site. This procedure may apply to situations where the user of the implantation system may consider a portion of the staple bridge to be separated too far from the opposing surface of the implantation location. Such situation may be referred to as having the staple bridge portion being proud of the implantation location. To address this situation, after staple inserter 3021 has been removed from staple S, tamp 2455 may be positioned to contact bridge portions 2461 (FIG. 29) when it is proud as shown in FIG. 30 (with the understanding that staple inserter 3021 would be first removed). The bottom surface of slot 2457 thus acts as a tamping surface, and sufficient distally oriented force is applied to tamp 2455 so that such tamping force advances the proud bridge toward the fracture.

In further variations and implementations, engagement surface 2435 may be formed to include other features to resist slipping or movement relative to contemplated locations of pilot holes. For example, the distal ends surrounding drill guide holes 2449 may have one or more barbs, point or other sharp edges oriented and configured to help prevent the drill guide 2421 from slipping once it is placed in operative position at the implantation site. As illustrated, serrations are formed at distal ends of the drill guide holes 2449.

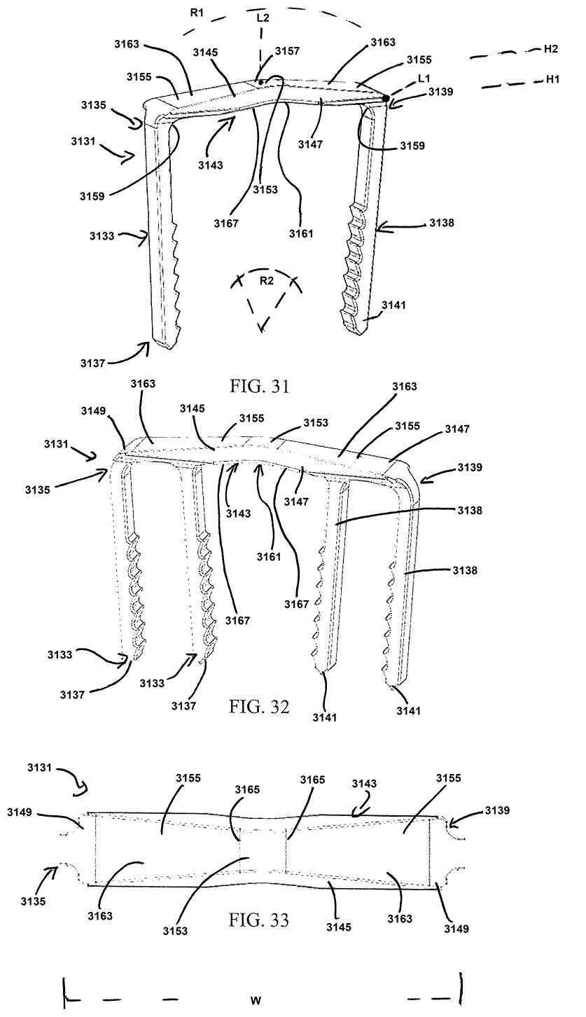

In addition to the surgical staple configurations discussed with reference to FIGS. 1 and 6-12, FIGS. 31-33 are views of still further implementations of surgical staple configurations according to the present disclosure. Surgical staples 3131 and 3131′ (FIGS. 31 and 32, respectively) include one or two pairs of legs 3133, 3138 configured and disposed so be on different sides of a fracture to be treated, such as a foot fracture. So, in the illustrated implementations, surgical staple 3133 has a single, first leg 3133 to be located on a first side of the fracture, and a single, second leg 3138 to be located on a second side of the fracture different from the first. Surgical staple 3131′ is a four-legged staple, with two first legs 3133 to be located on a first side of the fracture, and two second legs 3135 to be located on a second side of the fracture.

Surgical staples 3133, 3133′ may be made of any material suitable for surgical procedures, such as nitinol or other resilient, polymeric material capable of exerting inwardly directed force to cause compression of bone on either side of a fracture line being treated.

Legs 3133, 3138 have corresponding proximal ends 3135, 3139 and extend distally to terminate in respective tips 3137, 3141. A bridge 3143 has opposite bridge ends 3149 connect at the proximal ends 3135, 3139 of staple legs 3133, 3138 and top and bottom bridge portions 3145, 3147 extending therebetween. The outer portions of bridge ends 3149 connect to respective proximal ends of legs 3133, 3138 at location points L1, which correspond to a first maximum height H1 relative to the tips 3137, 3141 of the legs 3133, 3138, at respective, bridge ends 3149.

Bridge 3143 has an apex portion 3153 disposed between bridge ends 3149, such apex portion 3153 configured to have a second location point L2 corresponding to a second height H2 relative to the tips 3137, 3141 of legs 3133, 3138. In this implementation, top portion 3145 has a pair of planar surfaces 3155 defined as upper surfaces 3163 of top portion 3145. These upper, planar surfaces 3163 extend from respective bridge ends 3149 at height H1, inwardly and upwardly to apex portion 3153 which apex portion is at a height H2 greater than H1. As such, the upper planar surfaces on respective sides of apex portion 3153 for an angled bridge joint 3157 at apex portion 3153, which likewise is located to define a central bridge portion 3161 having lateral edges 3165.

The use of planar surfaces 3155, such as upper planar surfaces 3163, rather than certain arcuate surfaces, has been found to increase inwardly directed force exerted by the resiliency of the staple, especially as may be imparted by the bridge 3143 and transmitted down legs 3133, 3138. By way of illustration only, if upper surface of bridge 3143 were without planar surfaces 3155 and instead extended from first location points L1 through location point L2 in an arc with a constant radius, such reference curve would have a reference radius value R1. In contrast, by replacing such constant radius with planar surfaces 3155 at some or all of the connection between locations L1 and L2, as done by the above-described planar surface features of staples 3133, 3133″ such configuration causes bridge joint 3157 to have a joint radius value R2 smaller than reference radius value R1.

The smaller radius value R2 acts to increase inward compression after staples 3131, 3131′ have been inserted around a fracture because, as described in reference to previous staple embodiments and as applicable to staples 3131, 3131′, legs 3133, 3138 are spread slightly from their relaxed state upon insertion. The outward spread of staple legs is transmitted to bridge 3143 and its staple corners 3159, and the resiliency of the staple, especially at these bridge locations, creates inward or compressive force between legs 3133, 3138. Given the contribution to inward force from resiliency of bridge 3143, upon staple insertion, the tighter or smaller joint radius value R2 exerts greater inward compression than otherwise created by R1, per unit of outward linear deflection (spread) of legs 3133, 3138.

With these biomechanical aspects in mind, this disclosure encompasses variations in the number and arrangement of surfaces, whether planar as illustrated, or even arcuate, which such arrangements form tighter radius values than otherwise formed by the correspondingly larger radius values of arched or flat bridges which extend in larger or constant arcs between opposite staple legs. Accordingly, given a predetermined staple width W defined as the distance between staple corners 3159, in one suitable implementation, planar upper surfaces may be extended over at least 80% of such staple width.

In another alternative embodiment, instead of planar upper surfaces 3163 terminating at a pair of lateral edges 3165 and apex portion 3153 having a corresponding width between such lateral edges 3153, planar upper surfaces 3163 can extend inwardly to a single lateral edge such single edge having little to no width, and forming an angle at such junction sufficient to increase compression compared to the reference radius value discussed previously.

In still other alternative embodiments, bridge 3143 has a central bridge portion having a radius defined in either the upper or lower surface which is smaller than the reference radius value by virtue of such central bridge portion extending over no more than 20% of the referenced stable width.

A further embodiment may include two additional planar surfaces 3167 for a total of four planar surface 3155. The two additional planar surfaces 3167 are located on the bottom portion 3147 of bridge 3143 to form bottom planar surfaces. The additional planar surfaces 3167 extend upwardly and inwardly toward one another and extend below the pair of upper planar surfaces 3163 discussed previously located on top portion 3145 of bridge 3143.

Advantages of the foregoing apparatus and related methods are apparent from the foregoing disclosure.

With regard to staples 3131, 3131′, the planar portions may be formed in any of a variety of staple configurations, whether in-line, four-leg staples, or I-beam staples. The advantages of the planar portions may be included in staples with angle cuts as disclosed herein with reference to FIGS. 7-11.

With regard to drill guide 2421, its features have the advantage of minimizing movement or slipping relative to the implantation site. When the tamp 2455 is integrated into drill guide 2421, it serves as a two-in-one tool for associated staple implantation systems and procedures, allowing tamping of staples after insertion. This thus reduces the number of components needed for this surgery.

Drill guide head 2431 may be selectively attached and detached from drill guide handle 2423, for ease of maintenance, replacement, repair, and the like.

Having a detachable drill guide head 2431 likewise means that drill guide 2421 may be packaged as a kit, in which multiple drill guide heads of different configurations, for use with different staple types and configurations, may each be used selectively with the same drill guide handle.

Similarly, tamp 2455, when removably detachable from handle 2423, can likewise come as a kit of multiple tamps with different slot dimensions.

The ability to use offset k-wires with drill guide 2421 helps ensure the drilled pilot holes are parallel for staple insertion.

The offset k-wires may also be used to help the user locate where the pilot holes were drilled.

More accurate pilot hole location helps minimize the amount of insertion or force applied to bone at the implantation location, including when the staple gets tamped into place.

As a still further advantage, the drill guide disclosed herein may minimize or eliminate the need to re-drill pilot holes.

The features of the drill guide which help keep it in place may potentially reduce cognitive stress on the user by reducing the potential for inadvertent hand movements causing the drill guide to shift position.

As still another advantage, offset k-wires also improve assurances that all trajectories of the drilled holes are parallel to each other since using a pair of the off-set k-wires resist both rotating or rocking of the staple drill guide relative to the bone surface after drilling one of the pilot holes.

With regard to the staple inserter, small pilot holes can be more easily located, even after all drilling instrumentation has been removed, by having the offset k-wires received in the slots on the Staple Inserter, which in turn gives the user guidance to the pilot holes.

Still further such slots, when k-wires are received therein, help to get the staple in the correct trajectory that matches the trajectory of the pilot holes.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A hand-held drill guide for use in a surgical staple procedure on a patient with a fracture, the drill guide comprising:
a handle having opposite proximal and distal ends and a grasping surface extending between the ends, the grasping surfacing configured to be held by a user during the surgical staple procedure;
at least one head removably attachable to the handle at the distal end of the handle, the head having first and second head surfaces extending distally to define a drill guide tip;
wherein the first of the head surfaces comprises an engagement surface positionable in operative proximity to the fracture to be treated;
wherein the second of the head surfaces is oriented to define a user-accessible surface when the engagement surface is positioned in operate proximity to the fracture to be treated;
wherein the head has multiple bores extending between the engagement surface and the user-accessible surface, the bores having inner surfaces and surrounding head portions to define corresponding bore diameters;
wherein the bores comprise a first set of bores and a second set of bores,
wherein the first set of bores are sized to slidably and rotatably receive a surgical drill bit therethrough, the drill bit having a first hardness associated therewith;
wherein the inner surfaces of the first set of bores have a second hardness greater than the first hardness to protect the surrounding head portions from abrasion by the drill bit during the procedure;
wherein the bores of the first set are located in laterally spaced relation to each other along a first lateral axis of the head to define drill guide holes;
wherein the second set of bores is spaced longitudinally and in the proximal direction from the first set of bores, the second set of bores disposed in laterally spaced relation to each other along a second, lateral axis of the head, the second lateral axis parallel to the first lateral axis to define offset, k-wire holes located by an offset distance from the drill guide holes; and
wherein the k-wire holes are sized to slidably and rotatably receive k-wires therethrough.

2. The drill guide of claim 1, wherein the drill guide comprises multiple heads, the multiple heads comprising different configurations of the set of drill guide holes, a first of the heads comprising two of the drill guide holes corresponding to use with a two-leg staple, a second of the heads comprising four of the drill guide holes laterally disposed to correspond to a four-leg, in-line staple, and a third of the heads comprising four of the drill guide holes laterally and longitudinally disposed to correspond to a four-leg, I-beam staple.

3. The drill guide of claim 1, further comprising a tamp located at the proximal end of the handle, the tamp having portions defining a slot therein, the slot having walls dimensioned to receive bridge portions of surgical staples therein, wherein the slot is located relative to the grasping surface, so that, if the slot receives the bridge of the staple after insertion of the staple into the bone, distally directed force applied to the grasping surface is transmitted to the staple received in the slot.

4. A method of implanting a surgical staple in a bone-fracture-repair procedure, the method comprising:
determining an implantation location for the staple relative to the bone fracture;
selecting a staple leg configuration for the determined implantation location from a plurality of different, staple leg configurations;
positioning a set of drill guide holes of a drill guide corresponding to the staple leg configuration in operative proximity to the implantation location;
pinning the drill guide holes relative to the implantation location by fixing k-wires between the bone and the drill guide;
advancing a drill bit through the drill guide holes to form pilot holes corresponding to the staple to be implanted;
detecting a mark on the drill bit corresponding to the limit depth of the pilot hole and to the staple to be used;
ceasing to advance the drill bit into the bone in response to the detection of the mark at a predetermined position corresponding to the limit depth.

5. The method of claim 4, wherein the step of pinning the drill guide holes relative to the implantation location comprises advancing a set of the k-wires at respective locations spaced from the drill guide holes through a corresponding set of k-wire holes of the drill guide.

6. The method of claim 4, wherein the pinning step comprises fixing a first one of the k-wires between the bone and the drill guide by advancing the k-wire through a first one of the drill guide holes.

7. The method of claim 6, wherein the pinning step comprises fixing a second one of the k-wires between the bone and the drill guide by advancing the second one of the k-wires through a second one of the drill guide holes.

8. The method of claim 7, wherein the pinning step comprises advancing at least one of the k-wires through at least one pilot hole formed at the implantation location.

9. The method of claim 4, wherein the step of advancing the drill bit through the drill guide holes comprises forming the pilot holes along a first lateral axis on either side of the bone fracture, wherein the step of fixing the k-wires comprises fixing the k-wires in spaced relation to each other along a second lateral axis parallel to the first lateral axis, the distance between the two lateral axes defining an offset.

10. The method of claim 9, further comprising a step aligning the staple to be implanted with the pilot holes by reference to the offset.

11. The method of claim 10, wherein a step of aligning the staple comprises associating the staple with a staple inserter; and receiving distal portions of the k-wires into respective slots formed in the staple inserter.

12. The method of claim 11, further comprising applying distally oriented force to the staple associated with the inserter while the k-wires are received in the slots to implant the staple at the implantation location and to advance the legs through the pilot holes.

13. The method of claim 12, further comprising:
separating the staple from the staple inserter after the step of applying the distally oriented force;
positioning a tamping surface of the drill guide to contact a proud bridge of the staple after implantation of the staple, and;
applying tamping force to advance the proud of bridge toward the bone.

14. The method of claim 4, wherein the step of selecting the staple leg configuration comprises selecting a corresponding drill guide head from a plurality of drill guide heads of different drill guide hole configurations.

15. An implantable surgical staple for treating a foot fracture of a patient, comprising:
at least one, first leg configured to be disposed on a first side of the fracture, the first leg extending from a first proximal end to terminate distally in a first tip;
at least one, second leg configured to be disposed on a second side of the fracture different from the first side, the second leg extending from a second proximal end to terminate distally in a second tip; and
a bridge having top and bottom portions extending between the first leg and the second leg and having opposite bridge ends connected at the first and the second proximal ends of the legs at first location points corresponding to a first maximum height relative to the tips of the legs at the respective bridge ends;
wherein the bridge has an apex portion disposed between the bridge ends and having a second location point corresponding to a second height relative to the tips of the legs;
wherein the first location points of the bridge ends and the second location point of the apex portion defines a reference radius value when the points are interconnected by a reference curve of constant radius; and
wherein at least one of the top and the bottom portions of the bridge comprise a pair of planar surfaces extending upwardly between respective bridge ends and the apex portion, and inwardly toward the apex portion to meet at an angled bridge joint, the bridge joint defining a joint radius value smaller than the reference radius value.

16. The implantable surgical staple of claim 15, wherein the top and bottom portions of the bridge extend between the first leg and the second leg to define respective staple corners and a central bridge portion of the bridge, the central bridge portion located between the corners;
wherein the planar surfaces comprise two planar, upper surfaces disposed on the top portion and extending from respective staple corners to the central bridge portion.

17. The implantable surgical staple of claim 16, wherein the distance between the staple corners corresponds to a predetermined staple width, and wherein the planar upper surfaces extend over at least 80% of the staple width.

18. The implantable surgical staple of claim 17, wherein the central bridge portion extends no more than 20% of the staple width.

19. The implantable surgical staple of claim 18, wherein the planar surfaces extend inwardly to terminate at a pair of lateral edges adjacent to the apex portion of the bridge.

20. The implantable surgical staple of claim 16, comprising an additional two of the planar surfaces, the additional two, planar surfaces located on the bottom portion, extending upwardly and inwardly toward one another and extending below the pair of planar surfaces located on the top portion.
